(19) United States
(12) Patent Application Publication
(10) Pub. No.: US 2026/0315735 A1
(43) Pub. Date: Oct. 15, 2026

(54) IN-PACKAGE AROMA COMPOUND FOR MEDICAL DEVICE

(71) Applicant: Hollister Incorporated, Libertyville, IL (US)

(72) Inventor: Satwinder S. Panesar, Foxford (IE)

(21) Appl. No.: 19/113,707

(22) PCT Filed: Sep. 26, 2023
(86) PCT No.: PCT/US2023/075130

§ 371 Date: Mar. 20, 2025

Related U.S. Application Data
(60) Provisional application No. 63/377,259, filed on Sep. 27, 2022.

(51) Int. Cl. A61M 25/00; A61L 9/04; A61L 9/05

(52) U.S. Cl. CPC A61M 25/002; A61L 9/042; A61L 9/05; A61M 2205/588

(57) ABSTRACT

A medical device package (10) includes a case (12) containing a hydration medium (16), a medical device (14), and an aroma compound (18) §. The aroma compound is configured to transition from a liquid phase to a vapor phase and may be released from the case in the vapor phase. The aroma compound in the vapor phase is stored in a headspace of the case. A method of making the medical device package includes adding the aroma compound, the hydration medium and the medical device to the case, closing the case and exposing the case to an electron beam. The method may further include transitioning the aroma compound from a liquid phase to a vapor phase.

### TECHNICAL FIELD

The present disclosure generally relates to packaging for a medical device, and more particularly, to packaging for a urinary catheter, the packaging having an aroma compound which is released as a vapor upon opening of the package.

### BACKGROUND

A urinary catheter may be packaged in a case and provided to an end user. For catheterization, the user opens the case, removes the catheter and inserts the catheter into the urethra. After usage, the catheter is removed from the urethra and may be returned to the case for disposal.

Catheterization is often associated with bodily odors which may be perceived as unpleasant by some users, or otherwise cause the user to become self-conscious of the odor. For example, users commonly perceive a urine odor associated with removal of the catheter. Such bodily odors may also be perceived by the user while carrying the case to a location for disposal.

Accordingly, it may be desirable to provide a medical device package configured to reduce or negate the user's perception of bodily odors associated with use of the medical device. It may also be desirable to provide a medical device package configured to reduce or negate the user's perception of associated bodily odors after use of the medical device. Further, it may be desirable to provide a method making such a medical device package.

### SUMMARY

There are several aspects of the present subject matter.

In one aspect, a medical device package may include a case having an open end configured to be selectively opened and closed, wherein the case may be opened to allow access to an interior of the case and the case may be closed to prevent access to the interior of the case. The package may further include a hydration medium disposed in the case, a medical device disposed in the case, and an aroma compound disposed in the case. The aroma compound may be configured to transition from a liquid phase to a vapor phase within the case. Opening the case may allow for release of the aroma compound in the vapor phase.

In another aspect, a method of making a medical device package may include adding an aroma compound to a case, the case having an open end configured to be selectively opened and closed, wherein the case may be opened to allow access to the open end and an interior of the case and may be closed to prevent access to the open end and the interior of the case. The method may also include adding a hydration medium to the case and adding a medical device to the case. The method may further include closing the case and exposing the case to sterilizing radiation, such as an electron beam. The aroma compound may be added to the case in a liquid phase and may be configured to transition to a vapor phase. The case may include a headspace and the aroma compound, in the vapor phase, may be arranged in the headspace with the case closed.

In another aspect, a medical device package may include a case having an open end and a cap selectively movable relative to the open end, such that in an open condition of the case, the cap is removed from the open end to allow access to an interior of the case, and in a closed condition of the case, the cap is disposed on the open end to prevent access to the interior of the case. The package may further include a hydration medium disposed in the case, a urinary catheter disposed in the case, and a plurality of capsules containing an aroma compound liquid disposed in the case. The capsules have a capsule coating within which the aroma compound liquid is contained. The capsule coating is configured to liquify in the hydration medium to release the aroma compound liquid into the hydration medium with the case in the closed condition.

In yet another aspect, a urinary catheter package may include a case having an open end and a headspace formed within the case. The case may be configured to be selectively opened and closed at the open end. The package may further include a hydration medium disposed in the case, a urinary catheter disposed in the case and an aroma compound, in a vapor phase, contained in the headspace of the case with the case closed. The aroma compound may be released from the case when the case is opened, and the aroma compound may be formulated to have a predetermined aroma.

### BRIEF DESCRIPTION OF DRAWINGS

FIG. 1 is a perspective view showing an example of a medical device package according to the present embodiments;

FIGS. 2 and 2A are cross-sectional views of the medical device package of FIG. 1 including an enlarged section ‘A’, the medical device package depicted in a closed condition to contain an aroma compound liquid;

FIGS. 3 and 3A are cross-sectional views of the medical device package of FIG. 1 including an enlarged section ‘B’, the medical device package depicted in a closed condition to contain an aroma compound vapor;

FIG. 4 is a cross-sectional view of the medical device package of FIG. 1 in an open condition to release an aroma compound vapor; and

FIG. 5 is a diagram showing the medical device package of FIG. 1 exposed to radiation.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

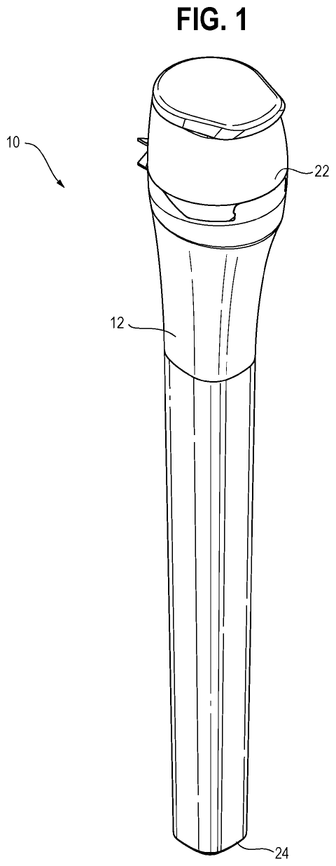

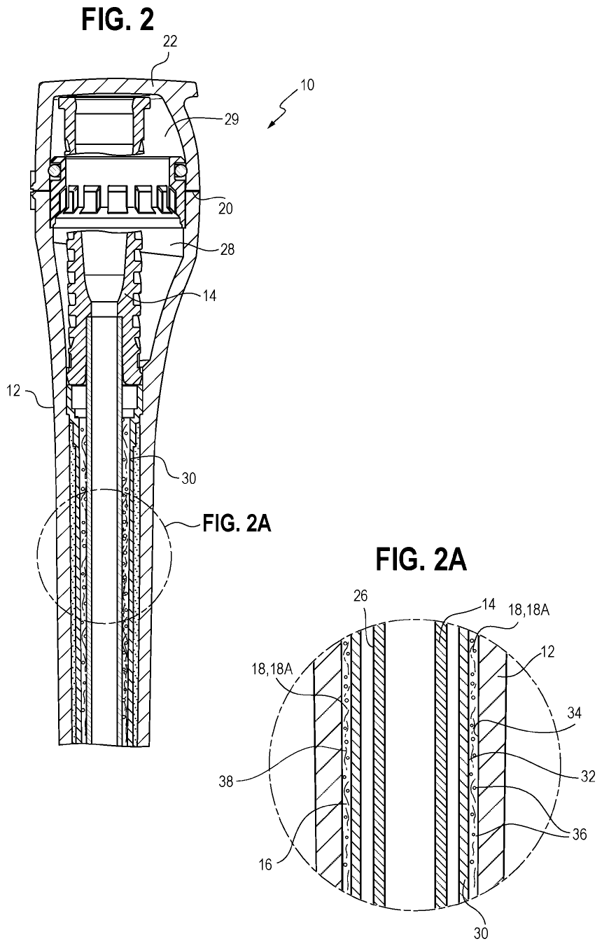

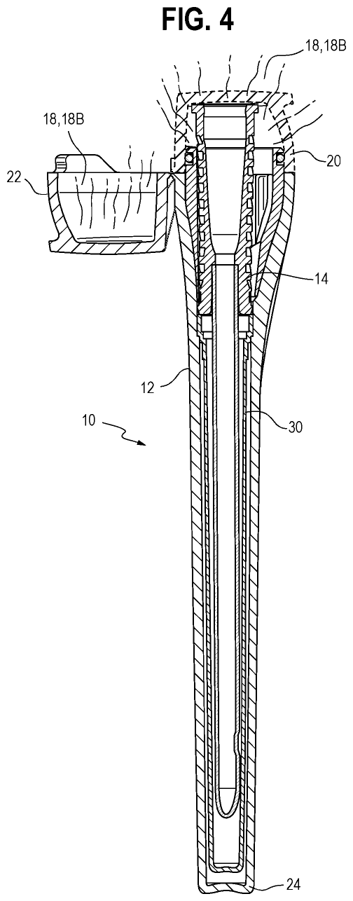

With reference to FIGS. 1, 2 and 2A, a medical device package 10 according to embodiments herein generally includes a case 12 and a medical device 14, a hydration medium 16 and an aroma compound 18 disposed in the case 12. The case 12 is configured to be selectively opened and closed, i.e., selectively placed in an open condition and in a closed condition. In one example, the case 12 includes an open end 20, such that the case 12 may be opened to allow access to the open end 20 and an interior of the case 12 (see FIG. 4), and may be closed to prevent access to the open end 20 and the interior of the case 12.

With reference to FIGS. 1, 2, 2A and 4, in one example, the case 12 includes a cap 22 configured for selective movement relative to the open end 20 to open and close the case 12. For example, to open the case 12, i.e., to place the case 12 in the open condition, the cap 22 may be removed from the open end 20 to allow access to the open end 20 and the interior of the case 12. Conversely, to close the case 12, i.e., to place the case 12 in the closed condition, the cap 22 is placed on the open end 20 to prevent access to the open end 20 and the interior. In the closed condition, the case 12 may be substantially sealed and a sterile environment may be provided within the case 12. To this end, the cap 22 may be secured to the open end 20 of the case 12 in any suitable manner so as to substantially seal the case 12.

According to an embodiment, the case 12 may be provided as an elongated tube-like structure having a closed end 24 opposite to the open end 20. For example, the case 12 may formed substantially as a tubular structure and may have any suitable cross-sectional shape viewed in the direction of a longitudinal axis. Examples of suitable cases are shown and described in WO2017/185052, which is incorporated herein by reference in its entirety.

The medical device 14 may include a hydrophilic layer 26 configured to absorb a portion of the hydration medium 16, which may then provide lubricity on the medical device 14. In one example, the medical device 14 is a urinary catheter. Lubricity of the hydrophilic layer 26 may provide additional comfort and ease of use during catheterization.

The hydration medium 16 partially fills the case 12 and a headspace 28 is formed in an unfilled portion of the case 12. The hydration medium 16 may be a water or aqueous solution suitable for hydrating or wetting the hydrophilic layer 26 or material of the medical device 14. Examples of suitable hydration mediums are described WO2019/222652 which is incorporated herein by reference in its entirety. Such hydration mediums may include, but are not limited to, a hydration foam or a hydration fluid including mucilage, deep eutectic fluid and/or oil.

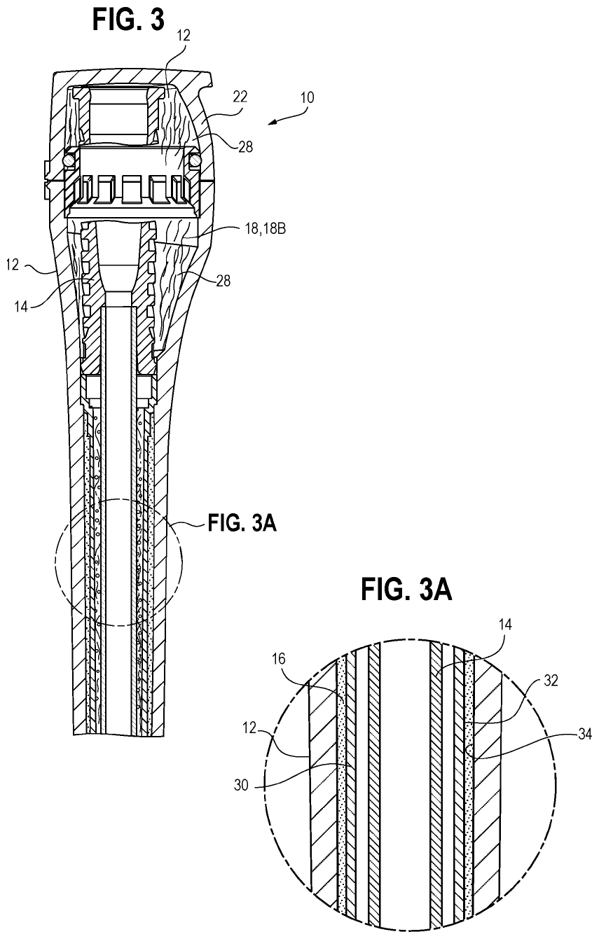

Referring now to FIGS. 2, 2A, 3 and 3A, the aroma compound 18 may be disposed within the case 12. The aroma compound 18 may initially be added to the case 12 in a liquid phase, i.e., as an aroma compound liquid 18A. In one example, the aroma compound 18 may be added to the hydration medium 16 (see FIGS. 2 and 2A). The aroma compound 18 is configured to transition from the liquid phase 18A to a vapor phase 18B, i.e., an aroma compound vapor 18B within the case 12. The aroma compound vapor 18B may be disposed substantially within the headspace 28 with the case 12 closed (see FIGS. 3 and 3A).

The medical device package 10, optionally, may further include a hydration liner 30 arranged within the case 12. The hydration liner 30 may be configured to receive a portion of the medical device 14. The hydration medium 16 may be contained between an outer wall 32 of the hydration liner 30 and an inner wall 34 of the case 12. The hydration liner 30 may also include a liquid impermeable/vapor permeable barrier (not shown). In this manner, the hydration medium 16, as a liquid, does not contact the portion the medical device 14 arranged in the hydration liner 30. However, a water vapor component of the hydration medium 16 may be transmitted through the barrier and absorbed by the hydrophilic layer 26 of the medical device 14. It will be appreciated that such a configuration is shown and described for illustrative purposes only, and that the present disclosure is not limited to such a configuration. For example, a hydration liner 30 may be omitted or a differently configured hydration liner may be provided. In some examples, the medical device 14, including the hydrophilic layer 26 may be directly exposed to the hydration medium 16.

In use, the aroma compound 18 may be added to the case 12 as the aroma compound liquid 18A, the case 12 may be closed, and the aroma compound liquid 18A may transition to the vapor phase 18B. The aroma compound vapor 18B may be disposed in the headspace 28. The medical device package 10 may be provided to an end user in this configuration. For catheterization, the user may open the case 12 to access the medical device 14, e.g., the urinary catheter. The aroma compound vapor 18B may be released from the case 12 when the case 12 is opened, for example, by removal of the cap 22 from the open end 20. The aroma compound 18 has an associated aroma (also referred to as a fragrance or perfume) which may be perceived by the user or others in a vicinity of the user at least during catheterization. The aroma compound 18 may be formulated such that the associated aroma perceived by the user reduces, negates or substantially negates bodily or other odors commonly associated with use of a urinary catheter.

Referring again to FIGS. 3, 3A and 4, at least a portion of the headspace 28 may be provided within the cap 22. Accordingly, at least a portion of the aroma compound vapor 18B may be disposed within the cap 22. In one example, as noted above, the cap 22 may be removed from the open end 20 to open the case 12. At least some portion of the aroma compound vapor 18B may remain in the headspace 28 portion within the cap 22. When use of the medical device 14 is complete, the user may return the medical device 14 to the case 12 and close the case 12 by returning the cap 22 to the open end 20. Accordingly, the portion of the aroma compound vapor 18B remaining in the cap 22 may be returned to the case 12. In this manner, the associated aroma may still be provided after use of the medical device 14 as well, and odors associated with the medical device 14 may be negated when the user transports the case 12 to another location, for example, to dispose of the medical device 14.

As shown in FIGS. 2 and 2A, the medical device package 10, optionally may include a plurality of capsules 36. The capsules 36 comprise a capsule coating 38 within which the aroma compound 18 may be contained in the liquid phase 18A. Thus, in one example, the aroma compound 18 may be added to the case 12, for example, to the hydration medium 16, in the capsules 36. In one example, the capsules 36, with encapsulated aroma compound 18 at less than 0.01% w/w, may be added to the case 12.

Referring again to FIGS. 3 and 3A, the capsule coating 38 may be configured to liquify to release the aroma compound 18 into the hydration medium 16. In one example, the capsule coating 38 may liquify in response to exposure to radiation 40, such as gamma radiation or an electron beam 40 (or “e-beam”), for example, during a sterilization process. For instance, after the capsules 36 are added to the case 12, the case 12 may be closed and sterilized by exposure to the electron beam 40 to sterilize the medical device 14. The aroma compound liquid 18a released from the capsules 36 may then transition to the aroma compound vapor 18B.

The capsule coating 38 may comprise a material sensitive to radiation exposure (e.g., gamma radiation or electron beam exposure), such as a polysaccharide. In one example, the capsule coating 38 may include a water-soluble anionic polysaccharide, such as gellan gum. The capsule coating 38 may be formed by dissolving gellan gum at 1-5% w/w in water at a temperature above 75 degrees Celsius to form a homogenous solution. The homogenous solution may be cooled below 38 degrees Celsius to then provide a hydrogel, and at greater than 2% w/w results in forming a hard gel or coating. Alternatively, the capsule coating 38 may include gellan gum and citric acid. The capsule coating 38 may be formed by dissolving the gellan gum at 1-5% in water at a temperature greater than 75 degrees Celsius to form a homogenous solution and adding citric acid to the solution at greater than 0.05-1.00% w/w. In one example, the citric acid may be added at about 0.1-0.2% w/w, which may result in formation of a hard coating at ambient temperature.

In another example, the capsule coating 38 may liquify in response to exposure to a change in pH of the hydration medium 16. For example, the pH of the hydration medium 16 may decrease in response to exposure to radiation (gamma or electron beam 40) in a sterilization process. In one example, the pH value of the hydration medium 16 may be reduced from about 6 to about 4 in response to exposure to the electron beam 40. Accordingly, the capsule coating 38 may liquify when the pH of the hydration medium 16 is reduced to release the aroma compound liquid 18A into the hydration medium 16. Subsequently, the aroma compound 18 may transition to the vapor phase 18B. A capsule coating 38 configured to liquify from exposure to a change in pH may include, but is not limited to, one or more of a protein, a peptide and/or a polyphenol.

In still another example, the aroma compound 18 may be added directly the case 12, for example, directly to the hydration medium 16, in the liquid phase 18A. The capsules 36 may be omitted in such an example.

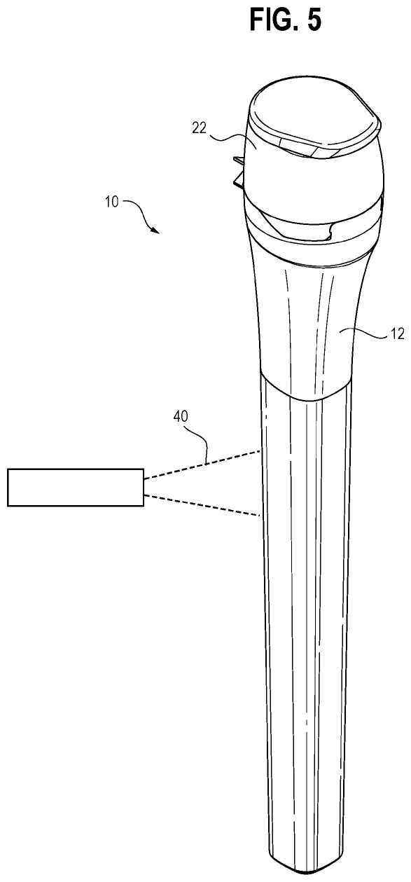

Referring to FIG. 5, and as noted above, the case 12 and the contents of the case 12, e.g., the medical device 14, may be sterilized in a sterilization process. The sterilization process is performed with the case 12 in the closed condition. In one example, the sterilization process may be controlled such that the case 12 and its contents are exposed to the electron beam 40 at about a 20-90 KGy radiation dose. In another example, the case 12 and the contents may be exposed to the electron beam 40 at about a 25-65 KGy radiation dose.

The aroma compound 18 may be formulated such that approximately 95%-100% of the aroma compound 18 transitions from the liquid phase 18A to the vapor phase 18B. Thus, approximately 95%-100% of the aroma compound 18 may be transitioned from the liquid phase 18A in the hydration medium 16 to the vapor phase 18B in the headspace 28. Accordingly, to the extent a portion of the aroma compound 18 may remain in the hydration medium 16 or is absorbed by the hydrophilic layer 26 after the phase transition, such a portion is negligible and should not impact catheterization.

Moreover, the aroma compound 18 may be selected to be a non-toxic compound and is provided to the case 12 in a relatively low concentration, for example, less than approximately 0.01% w/w. Thus, to the extent a portion of the aroma compound 18 may be absorbed by the hydrophilic layer 26, such a portion of the aroma compound 18 should not irritate the user's skin or urethra during catheterization due to the selection of the non-toxic compound and the relatively low concentration of the aroma compound 18. Accordingly, the aroma compound 18 may have negligible bio-toxicity impacts.

The aroma compound 18 may be an organic compound, and in further examples, a volatile organic compound. The volatile organic compound has a relatively low boiling point, for example, approximately at ambient temperature (e.g., approximate ambient room temperature). Thus, the aroma compound 18 may transition from the liquid phase 18A to the vapor phase 18B in response to reaching the ambient temperature.

The aroma compound 18 may include either a natural compound or a synthetic compound. It will be appreciated, that the aroma compound 18 may include a combination of one or more of the characteristics in the examples above, and/or be provided as a formulation including one or more components having one or more of the characteristics above.

The aroma compound 18 may include an ester. The ester may include one or more of geranyl acetate, methyl butyrate, ethyl acetate, isoamyl acetate and/or benzyl acetate.

The aroma compound 18 may include a terpene. The terpene may include one or more of geraniol, geranium citral, citronellol, linalool, limonene, camphor, carvone and/or eucalyptol.

The aroma compound 18 may include an amine. The amine may include one or more of trimethylamine, putrescine, cadaverine, indole and/or skatole.

The aroma compound 18 may include an aromatic. The aromatic may include one or more of eugenol, cinnamaldehyde, benzaldehyde, vanillin and/or thymol.

The aroma compound 18 may include an aldehyde. The aldehyde may include one or more of hexanal and/or isovaleraldehyde.

The aroma compound 18 may include an alcohol. The alcohol may include menthol.

The aroma compound 18 may include a thiol. The thiol may include one or more of benzyl mercaptan, allyl thiol, (menthlythio)methanethiol and/or ethyl-mercaptan.

The aroma compound 18 may include a ketone. The ketone may include one or more of 6-acetyl-2,3,4,5-tetrahydropyridine and/or oct-1-en-3-one.

The aroma compound 18 may include a lactone. The lactone may include one or more of gamma-nonalactone and/or gamma-decalactone.

In some examples, the aroma compound 18 may include 2,4,6-trichloroanisole.

In some examples, the aroma compound 18 may include diacetyl.

In some examples, the aroma compound 18 may include methylphosphine.

It will be appreciated that the aroma compound 18 may include a combination of components as well, such as those described in the examples above. That is, the aroma compound 18 may include, one or more of an ester, a terpene, an amine, an aromatic, an aldehyde, an alcohol, a thiol, a ketone, a lactone, 2,4,6-trichloroanisole, diacetyl, and/or methylphosphine.

According to present examples, the aroma compound 18 may be released from the case 12 as a vapor 18B upon opening the case 12, and have an associated aroma which may be perceived by the user as a pleasant aroma during use of the urinary catheter 14. Thus, the associated aroma of the aroma compound 18 may negate odors associated with use a urinary catheter commonly perceived to be unpleasant.

A method of making the medical device package 10 of the examples above may include adding the hydration medium 16 to the case 12, adding the aroma compound 18 to case 12, and adding the medical device 14 to the case 12. The method may also include closing the case 12 and exposing the case 12 to the electron beam 40, for example, in the sterilization process. The method may further include transitioning the aroma compound 18 from the liquid phase 18A to the vapor phase 18B to be stored in the headspace 28 of the case 12. It will be appreciated that in the following examples, references to the features above, identified by like terminology and/or like reference numbers, indicate that such previously-described features may be implemented in the following examples. Accordingly, further description of such features may be omitted below.

In examples of the present method, adding the aroma compound 18 to the case 12 may include includes adding the plurality of capsules 36 comprising the capsule coating 38 to the case 12, wherein the aroma compound 18 is contained within the capsule coating 38. The capsules 36 may be added to the hydration medium 16, or vice versa. Alternatively, adding the aroma compound 18 to case 12 may include adding the aroma compound 18 in the liquid phase 18A directly to the case 12. For example, the aroma compound liquid 18A may be added directly to the hydration medium 16, or vice versa. It will be appreciated that in some examples, the aroma compound 18 may be added to the hydration medium 16, or vice versa, prior to being added to the case 12.

As previously indicated, the medical device 14 may be a urinary catheter. Accordingly, adding the medical device 14 to the case 12 may include adding the urinary catheter 14 to the case 12.

Closing the case 12 may include closing the open end 20 of the case 12. Closing the open end 20 of the case 12 may include placing the cap 22 over the open end 20. Placing the cap 22 over the open end 20 may prevent access to the open end 20 and to the interior of the case 12, and further, may provide a substantially sealed environment within the case 12.

Transitioning the aroma compound 18 from the liquid phase 18A to the vapor phase 18B may occur with case 12 in the closed condition, such that the aroma compound vapor 18B is substantially provided in the headspace 28. At least a portion of the headspace 28 may be provided within the cap 22, such that at least a portion of the aroma compound vapor 18B may be provided within the cap 22.

Transitioning the aroma compound 18 from the liquid phase 18A to the vapor phase 18B may include increasing a temperature of the aroma compound liquid 18A to the boiling point of the aroma compound 18 which, in one example, is at or near an ambient temperature, i.e., a typical room temperature.

Transitioning the aroma compound 18 from the liquid phase 18A to the vapor phase 18B may include transitioning approximately 95%-100% of the aroma compound 18 to the vapor phase 18B. That is, in some examples, the aroma compound 18 may be selected have properties which promote or otherwise allow the transition of approximately 95% to 100% of the aroma compound 18 from the liquid phase 18A to the vapor phase 18B at ambient temperatures.

For catheterization, a user may open the case 12, for example, by removing the cap 22 from the open end 20. With the case 12 opened, at least a portion of the aroma compound vapor 18B may be released from the case 12 and the user may access the medical device 14 within the case 12.

In some examples, at least a portion of the aroma compound vapor 18B may be retained in the cap 22 after opening the case 12. Alternatively, or in addition, a portion of the aroma compound 18 may remain in the liquid phase 18A in the hydration medium 16. After using the medical device 14, e.g., the urinary catheter 14, the user may return the catheter to the case 12 and close the case 12, for example, by replacing the cap 22 over the open end 20. The portion of the aroma compound vapor 18B retained in the cap 22 may be returned to the case 12 in this manner. Alternatively, or in addition, portions of aroma compound liquid 18A remaining in the hydration medium 16 may be transitioned to aroma compound vapor 18B over time or with an increase in temperature. In this manner, the aroma associated with the aroma compound 18 may be provided after using the catheter 14, for example, during transport of the case 12 to another location for disposal.

Exposing the case 12 to radiation (such as gamma radiation or the electron beam 40) may be performed, for example, in the sterilization process. In this manner, the contents of the case 12, including the medical device or urinary catheter 14 may be sterilized. Exposing the case 12 to gamma radiation or the electron beam 40 may include exposing the case 12 to a radiation dose of approximately 20-90 KGy. In a further example, the case 12 may be exposed to a radiation dose of approximate 25-65 KGy.

As indicated above, in some examples, the aroma compound 18 may be provided to the case 12 in capsules 36 having capsule coating 38. The capsule coating 38 may liquify in response to exposure to the electron beam 40. Thus, exposing the case 12 to the electron beam 40 may include exposing the capsule coating 38 to the electron beam 40 within the case 12 to liquify the capsule coating 38. In this manner, the aroma compound liquid 18A may be released into the hydration medium 16, and subsequently transitioned to the vapor phase 18B.

Alternatively, or in addition, the capsule coating 38 may be reactive to a change in pH value. As previously described, the pH of the hydration medium 16 may decrease in response to exposure to radiation (gamma radiation or the electron beam 40). Thus, exposing the case 12 to the electron beam 40 may include exposing the hydration medium 16 to the electron beam 40 to reduce the pH of the hydration medium 16. In this manner, the exposure to the reduced pH hydration medium 16 may liquify the capsule coating 38 to release the aroma compound liquid 18A to the hydration medium 16, and subsequently transition to the vapor phase 18B.

The capsule coating 38 may include polysaccharide, and in one example, may include gellan gum. The method may further include forming the capsule coating 38 with the gellan gum, which in turn may include dissolving the gellan gum at 1-5% w/w in water at a temperature greater than 75 degrees Celsius to form a homogenous solution, and cooling the homogenous solution below 38 degrees Celsius. The cooled homogenous solution may result in formation of a hydrogel, and at greater than 2% w/w, results in forming the coating 38 as a hard gel.

In another example, the capsule coating 38 may include gellan gum and citric acid. In such an example, forming the capsule coating 38 may include dissolving the gellan gum at 1-5% in water at a temperature greater than 75 degrees Celsius to form a homogenous solution and adding the citric acid to the solution at greater than 0.05%-1.00% w/w. In one example, the citric acid may be added at about 0.1-0.2% w/w, which may result in formation of a hard coating at ambient temperature.

Accordingly, in the present embodiments of the medical device package 10, the aroma compound 18 in the form of liquid or capsules containing the liquid may be added to the case 12, along with the medical device 14 and hydration medium 16. A hydration liner 30 may be provided within the case 12 as well. The case 12 may be closed, for example, by securing the cap 22 at the open end 20. The case 12 may then be exposed to an electron beam 40, for example, in a sterilization process. Exposure to the electron beam 40 may cause the capsule coating 38 to liquify and release the aroma compound 18 to the hydration medium 16. Alternatively or additionally, exposure to the electron beam 40 may reduce the pH of the hydration medium which, in turn, may cause the capsule coating 38 to liquify and release the aroma compound 18 to the hydration medium 16. The aroma compound liquid 18A may transition to the aroma compound vapor 18B, which may be contained in the headspace 28 of the case 12. The medical device package 10 may be provided to an end user, for example, as a urinary catheter package.

For catheterization, the user may open the case 12, for example, by removing the cap 22 from the open end 20. Opening the case 12 allows for release of the aroma compound vapor 18B to the atmosphere and the associated aroma may be perceived by the user. The user may access the urinary catheter 14 in the case 12 through the open end 20. The aroma compound 18 is formulated for a timed release of the associated aroma, for example, approximately 10-15 minutes, corresponding to a typical usage time of the catheter 14. That catheter 14 may be returned to the case 12 after use and the user may transport the case 12 to another location for disposal. The aroma compound 18 retained the cap 22 and/or within the case 12 may continue to provide the associated aroma which may be perceived by the user while transporting the case 12 for example, to dispose of the case.

The aroma compound 18 may be selected so as to have no negative toxicity impact on the urethra during catheterization. The user may experience the presence of the aroma or fragrance upon opening the case 12 and during catheterization. Moreover, because an amount of the aroma compound 18 remaining in the hydration medium 16 is negligible, the remaining aroma compound 18, if any, has a negligible bio-toxicity impact on catheterization. Further, the aroma compound 18 is selected to have no skin/urethra bio-toxicity.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A urinary catheter package comprising:
a case having an open end configured to be selectively opened and closed, wherein the case is opened to allow access to the open end and an interior of the case, and is closed to prevent access to the open end and the interior of the case;
a hydration medium disposed in the case;
a urinary catheter disposed in the case; and
an aroma compound disposed in the case and configured to transition from a liquid phase to a vapor phase,
wherein opening the case allows for release of the aroma compound, in the vapor phase, from the case.

2. The urinary catheter package of claim 1, wherein the case is an elongated tube-like structure further comprising:
a cap selectively movable relative to the open end, such that the cap is removed from the open end to open the case and the cap is placed on the open end to close the case.

3. The urinary catheter package of claim 2, wherein the case includes a headspace and the aroma compound, in the vapor phase, is arranged in the headspace.

4. The urinary catheter package of claim 3, wherein at least a portion of the headspace is formed in the cap.

5. The urinary catheter package of claim 4, wherein at least a portion of the aroma compound, in the vapor phase, is retained in the cap after removal of the cap from the open end.

6. The urinary catheter package of claim 1, wherein approximately 95%-100% of the aroma compound transitions to the vapor phase.

7. The urinary catheter package of claim 1, wherein the aroma compound transitions from the liquid phase to the vapor phase at about ambient temperature.

8. (canceled)

9. The urinary catheter package of claim 1, wherein the urinary catheter comprises a hydrophilic layer configured to absorb at least a portion of the hydration medium.

10. The urinary catheter package of claim 1, further comprising a plurality of capsules comprising a capsule coating within which the aroma compound is contained, wherein the aroma compound is disposed in the case within the capsules.

11. The urinary catheter package of claim 10, wherein the capsule coating liquifies in response to exposure to an electron beam.

12. The urinary catheter package of claim 11, wherein the aroma compound is released into the hydration medium in response to liquifying the capsule coating.

13. The urinary catheter package of claim 10, wherein a pH value of the hydration medium is reduced in response to exposure to radiation.

14. The urinary catheter package of claim 13, wherein the capsule coating liquifies in response to exposure to the reduced pH value of the hydration medium.

15. The urinary catheter package of claim 14, wherein the aroma compound is released into the hydration medium in response to liquifying the capsule coating.

16. The urinary catheter package of claim 10, wherein the capsule coating comprises a polysaccharide.

17. The urinary catheter package of claim 16, wherein the polysaccharide comprises gellan gum.

18.-35. (canceled)

36. The urinary catheter package of claim 1, wherein the aroma compound comprises one or more of aldehyde, ester, terpene, amine, aromatic, alcohol, thiol, ketone, lactone, 2,4,6-trichloroanisole, diacetyl and methylphosphine.

37.-48. (canceled)

49. A method making a urinary catheter package, the method comprising:
adding an aroma compound to a case, the case having an open end configured to selectively opened and closed, wherein the case is opened to allow access to the open end and an interior of the case and is closed to prevent access to the open end and the interior of the case;
adding a hydration medium to the case;
adding a medical device to the case;
closing the case; and
exposing the case to radiation,
wherein the aroma compound is added to the case in a liquid phase and is configured to transition to a vapor phase, and
wherein the case further includes a headspace and the aroma compound, in the vapor phase, is arranged in the headspace with the case closed.

50.-96. (canceled)

97. A medical device package comprising:
a case having an open end and a cap selectively movable relative to the open end, such that in an open condition of the case, the cap is removed from the open end to allow access to an interior of the case, and in a closed condition of the case, the cap is disposed on the open end to prevent access to the interior of the case;
a hydration medium disposed in the case;
a medical device disposed in the case; and
a plurality of capsules containing an aroma compound liquid disposed in the case, the capsules comprising a capsule coating within which the aroma compound liquid is contained,
wherein the capsule coating is configured to liquify in the hydration medium to release the aroma compound liquid into the hydration medium with case in the closed condition.

98.-102. (canceled)
